(19) United States
(12) Patent Application Publication
(10) Pub. No.: US 2007/0264289 A1
(43) Pub. Date: Nov. 15, 2007

(54) COMPOSITIONS AND METHODS OF ADMINISTERING DOXEPIN TO MUCOSAL TISSUE

(75) Inventors: Hal J. Oien, Tualatin, OR (US); Edmond Truelove, Seattle, WA (US); Joel Epstein, Wilmette, IL (US)

Correspondence Address: MARSH, FISCHMANN & BREYFOGLE LLP, 3151 SOUTH VAUGHN WAY, SUITE 411, AURORA, CO, 80014, US

(73) Assignee: EOT Research Inc., Seattle, WA (US)

(21) Appl. No.: 11/684,515

(22) Filed: Mar. 9, 2007

Related U.S. Application Data
(62) Division of application No. 10/868,505, filed on Jun. 14, 2004, now abandoned.
(60) Provisional application No. 60/478,438, filed on Jun. 12, 2003.

(51) Int. Cl. A61K 31/335; A61K 31/195; A61K 47/14; A61K 9/10; A61P 43/00

(52) U.S. Cl. 424/400; 514/450; 514/562; 514/785

(57) ABSTRACT

Compositions and methods for pain relief involve delivery of doxepin to mucosal tissue. Vehicles for administering doxepin may be formulated for sustained release and/or site-specific application to maximize beneficial pain relief locally while minimizing significant adverse side effects.

### CROSS-REFERENCE TO RELATED APPLICATION

This application claims priority under 35 U.S.C. §119 from U.S. Provisional Patent Application Ser. No. 60/478,438, filed Jun. 12, 2003, which is incorporated herein by reference in its entirety for all purposes.

### FIELD

The invention relates to compositions and methods of dispensing doxepin compositions to mucosal tissue, particularly in topical vehicles for sustained pain relief.

### BACKGROUND

Doxepin is a tricyclic antidepressant drug. It is a dibenzexipin tricyclic compound (N,N-dimethyldibenz(b,e)oxepin-propylamine hydrochloride) with a formula of C₁₉H₂₁N.HCl, and has a molecular weight of 316. The action of the tricyclics appears to be both central and peripheral. The primary mechanism of action may be by affect on descending pathways by blocking reuptake of serotonin and nor-epinephrine. In the periphery, activity may relate to adenosine receptors. Doxepin has potent H1 and H2 receptor blocking activity. Recently, nonspecific enkephalin-like activity, not affecting beta-endorphins has been demonstrated in patients prescribed doxepin. The tricyclics may affect the NMDA receptor in addition to effects on the descending norepinephrine and serotinergic systems. Due to the role of NMDA-receptor-medicated effects in spinal nociception, the modulation of the NMDA receptor was studied and acetylcholine release was seen by tricyclics including doxepin, by non-competitive antagonism, suggesting that at least some of the effects of tricycles may be due to inhibition of spinal NMDA receptors, in addition to the action via monoaminergic transmission in the spinal cord.

Once systemically absorbed, doxepin is converted in the liver to desmethyldoxepin, which is an active metabolite. The metabolites are excreted in the urine following glucuronidation. Desmethyldoxepin has a half-life of 28-52 hours. Plasma levels of drug and metabolite are highly variable and correlate poorly with systemic dosing.

Systemic doxepin produces drowsiness in a significant number of patients at target plasma therapeutic ranges of plasma for treatment of depression of 30-150 ng/ml. Doxepin is contraindicated in patients with narrow angle glaucoma, or for those with urinary retention. The sedating effect of alcohol and other drugs may be potentiated by doxepin. Serious drug reactions may occur with MAO inhibitors. Cimetidine has been reported to result in higher than expected serum levels of tricyclic antidepressants (TCAs) in blood.

Doxepin is used in the management of depression and chronic pain. Systemic use leads to sleep facilitation, and pain effect, in addition to treatment of depression. Tricyclics have analgesic effects in neuropathic pain, independent of their antidepressant effect. Tricyclic antidepressants are commonly used in the management of chronic pain in low (<50 mg/day) to intermediate doses (50-150 mg/day). One review of multidisciplinary pain clinics reported use in 25% of patients with chronic pain. In another study, 36 patients with back/or neck pain and depression were treated in a placebo-controlled study and doxepin was documented to be effective in managing pain and depression. Doxepin has been used in combination with nonsteroidal analgesics in management of pain associated with advanced cancer. Systemic doxepin has been reported for use in pain management associated with stomatitis. Oral doxepin rinse has been reported to provide pain relief in patients with oral mucosal lesions due to cancer or cancer therapy. However, some patients who used an oral doxepin rinse developed adverse systemic side effects such as sedation or fatigue.

### SUMMARY

Various methods and modes of administering doxepin to relieve pain associated with mucosal tissue in a patient are described. For example, doxepin may be administered site-specifically to a mucosal region in a patient's mouth. Alternatively, doxepin may be administered topically to other mucosal tissues in other parts of the body such as ear, nose, throat, eye, genitourinary, and gastrointestinal mucosa. Doxepin may also be administered in a time-release vehicle formulated to sustain pain relief without causing significant adverse side effects such as drowsiness or sedation.

### BRIEF DESCRIPTION OF THE FIGURES

FIG. 1 is a schematic view of a delivery vehicle dispensing doxepin site-specifically to a mucosal region.

FIG. 2 is a schematic sectional view of a multi-layered doxepin delivery vehicle.

### DETAILED DESCRIPTION

Particular methods and modes of administering doxepin to mucosal tissue are described below. However, it will be appreciated that many additional formulas and manners of a administering doxepin to relieve pain associated with mucosal tissue are suggested and enabled by the description.

Doxepin may be administered site-specifically to relieve pain associated with particular mucosal regions in a patient's mouth. Doxepin may also be administered in a time-release manner to maximize its sustained effect while minimizing adverse side effects. Doxepin may be administered in a variety of vehicles such as ointment, gel, foam, film, powder, gum, lozenge, or tablet, among others. The vehicle may be atomized or formulated for dispensing in an aerosol form. Doxepin may be compounded with the vehicle in a pharmaceutically effective concentration, for example, between about 0.1% (w/w) and 5.0% (w/w). For some applications the doxepin concentration is most effective between about 0.1% (w/w) and 1.0% (w/w). Doxepin may be used to provide longer pain relief compared to topical anesthetic agents that have been used in the past. For example, some topical doxepin formulas may provide pain relief for more than 30 minutes. Other formulas may provide pain relief for even longer periods such as 1 hour, 3 hours, or more than 4 hours. Doxepin may be advantageously used for pain relief in mucosal tissues other than in the mouth, for example, ear, nose, throat, eye, genitourinary, and gastrointestinal mucosa (e.g.: foam, suppositories, etc.)

Doxepin may be incorporated advantageously in many possible vehicle forms. For example, doxepin may be incorporated in a slowly dissolving water soluble carrier strip. The strip may be applied to a target location in the mouth. The strip may be formulated to control sustained release of doxepin. The strip may be in the form of a single homogeneous sheet or film. Alternatively, the strip may have multiple layers with each layer having a different formulation, different drug compositions, different dissolution times, etc. The strip may have other ingredients such as plasticizers, flavoring agents, antimicrobial agents, adhesion components, etc. The strips/sheets may be formulated to deliver the drug primarily to the area where the strip adheres. Doxepin strips may be supplied in a continuous tape form. Doxepin dosage may be controlled or selected according to the surface area of the applied tape, or alternatively may correspond to tape thickness.

A doxepin strip or sheet, as described above, may be applied to a mucosal tissue region in an individual. The sheet may be comprised of a water soluble polymer and doxepin at a concentration sufficient to be absorbed through the mucosal tissue and to have a desired biological effect such as sustained pain relief.

The strip may be quite thin and flexible so that it dispenses doxepin in the mouth for an extended period with minimal notice or distraction to the individual. A strip may also be formulated to treat wounds such as cold sores, mucositis, or to help control post-surgical bleeding. The film may be formulated to increase or decrease adhesion to skin and mucosa. It may be adjusted by thickness and/or formulation to control the rate of dissolution. These features allow for specific vehicle designs required to place doxepin sources in specific mucosal regions and keep them there for specified amounts of time. The combination of dissolution rate, concentration of medication in the film, film size and shape all may contribute to the rate of administration. The rate can be specified and the dry film medication designed and produced to meet that specification. Films may be gamma radiation processed for sterilization as needed. Dispensing sheets may be manufactured by wet casting or extruding processes, for example, wet extruding at low temperature and pressure or dry extruding at high temperature and pressure.

Layered films may dispense unidirectionally, meaning that active ingredients are layered from the mucosa side to a neutral top layer. Alternatively, a film may dispense bidirectionally with the same or different active agents on opposite sides of the film.

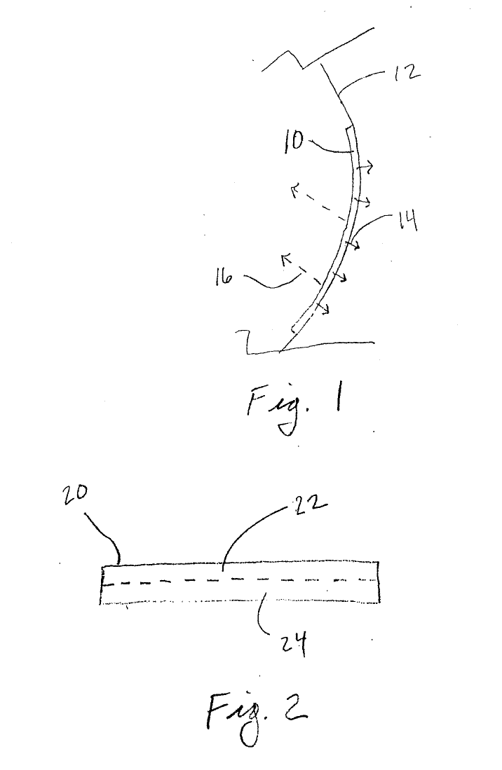

FIG. 1 shows strip 10 adhering to mucosal surface 12. Arrows 14 show the direction of doxepin permeation of mucosal tissue. Dashed line arrows 16 show the dispensing direction of the same or a different active component into the intraoral cavity. FIG. 2 shows a multilayer sheet in which layers 22 and 24 may be formulated for different purposes.

Doxepin may also be dispensed advantageously in combination with other drugs. Examples of biologically active substances that may be administered in conjunction with doxepin may include lidocaine, benzocaine, dyphenhydramine, and amitriptyline. Other topical treatments for mucosal disease (infections, reactive, autoimmune, mucositis, viral lesions, post surgical and post traumatic neuropathy, hemorrhage, stomatitis, etc.) which may be combined with doxepin include: antibiotics/antibacterials—tetracycline, chlorhexidine, metronidazol; iodine containing compounds, chlorine dioxide; antifungals—mycostatin, chlortrimazol, fluconazole, amphotericin, etc.; antivirals—acyclovir, interferon; steroids—hydrocortisone, all types and strengths of steroids, etc.; Vitamin A and other retinoids for treatment of dysplasia; azothioprine and other immune modulating medications; Tagamet—topical immune modulator; topical antineoplastic drugs—methotrexate; topical sclerosing agents; and anti-inflammatory agents. Other topical doxepin formulas may include gabapentin, clonidine, capsaicin, ginger, vitamins, buffering compounds—sodium bicarbonate, calcium, calcium carbonate, etc., coating compounds—sucrafate, eugenol, vitamin K, cocaine—hemostasis, morphine—pain control, and vitamin E.

Topical Application of doxepin with other medications for systemic absorption and effect through oral mucosa (analgesics, anxiolytics, beta blockers, nitroglycerin, hormones—estrogen etc, nicotine, sedatives and hypnotics) may include: morphine, synthetic opoid analgesics, diazepam, lorazapam, alprazolam, trialozam, propanolol, atenolol, nitroglycerin, estrogen, progesterone, testosterone, nicotine, and antihistamines.

Topical doxepin may also be compounded with one or more other analgesics, for example, acetaminophen, methyl salicylate, monoglycol salicylate, aspirin, mefenamic acid, flufenamic acid, indomethacin, diclofenac, alclofenac, diclofenac sodium, ibuprofen, ketoprofen, naproxen, pranoprofen, fenoprofen, sulindac, fenclofenac, clidanac, flurbiprofen, fentiazac, bufexarnac, piroxicam, phenylbutazone, oxyphenbutazone, clofezone, pentazocine, mepirizole, tiaramide hydrochloride, etc. Examples of steroidal anti-inflammatory agents which may be used in conjunction with doxepin include hydrocortisone, predonisolone, dexamethasone, triamcinolone acetonide, fluocinolone acetonide, hydrocortisone acetate, predonisolone acetate, methylpredonisolone, dexamethasone acetate, betamethasone, betamethasone valerate, flumetasone, fluorometholone, beclomethasone diproprionate, etc. Doxepin formulations may also include opioids for severe pain.

Cytokines or growth factors such as epidermal growth factors, or vascular-endothelial growth factors may also be included.

Doxepin may be applied to mucosal tissue in “time-release” formulations for sustained pain relief. “Time release,” as used herein, refers to “sustained release” or prolonged release of doxepin to mucosal tissue over an extended time period from a composition including doxepin and a vehicle. Accordingly, the composition may serve as a doxepin “reservoir” or “source” from which doxepin may be released gradually over the course of minutes, hours, or even days. Such gradual release may provide a sustained action of doxepin, with improved control of doxepin levels, stronger local effects, and less systemic exposure. Time release may be provided with a vehicle configured to remain substantially localized adjacent a mucosal tissue (or adjacent a selected mucosal region within the tissue) after placement of the vehicle (and doxepin) near the tissue (or region). Exemplary vehicles for time release may include solids (powders, crystals, capsules, etc.), gels, pastes, foams, viscous/sticky solutions, etc. Vehicles configured for time release may remain near the mucosal tissue/region for any suitable time period, but generally at least five or ten minutes. For some applications a vehicle is formulated to release doxepin for at least several hours.

### EXAMPLE 1

### Gels Including Doxepin

This example describes exemplary gels that may include doxepin. A gel, as used herein, is a viscous, semi-solid composition provided by a solid network holding liquid. The solid network may be a network of associated, entangled, and/or covalently linked aggregates, particles, and/or molecules, among others. Gels may be used to target extended or prolonged delivery of doxepin to a specific tissue site, such as a selected mucosal region within the mouth.

The gel may be a thixotropic gel, which is a gel that flows more readily in response to agitation and/or an applied shear stress (such as when stirred, shaken, or brushed onto a surface) and that returns to a less flowable form after the agitation and/or stress is removed. Accordingly, a thixotropic gel may have a viscosity that can be decreased before and/or during application of the gel, and that increases after application, for local retention of the applied gel. Thixotropic gels may achieve superior penetration and increased surface area contact and therefore improved uptake of doxepin. For example, thixotropic gels applied to the oral mucosa may spread until they reach a low pressure state at which point they may gel in place. This behavior may increase substantivity (longevity of clinical effectiveness) by reducing displacement of the gel by pressure.

Gels may include an amount of a gelling agent effective to form a composition for topical application. Exemplary concentrations of gelling agents are from about 0.1% to 20% by weight, or about 0.5% to 5% by weight. Gelling agents may include, among others, carboxypolymethylene, Veegum®, poloxamers, carrageenan, Irish moss, gums (such as gum karaya, gum arabic, gum tragacanth, xanthan gum, etc.), starch, alginate, polyvinylpyrrolidone, celluloses (such as hydroxyethyl propylcellulose, hydroxybutyl methyl cellulose, hydroxypropyl methyl cellulose, hydroxyethyl cellulose, carboxymethyl cellulose, carboxypropyl cellulose, and/or the like), carboxyvinyl polymers, and/or other hydroxyvinyl polymers. Other exemplary gelling agents may include Carbopol® polymers, colloidal silica, and/or complex colloidal magnesium aluminum silicates, to form thixotropic gels.

Gels may include any suitable solvents. Gels may be aqueous, that is, including water as at least the major solvent and/or the major component.

### EXAMPLE 2

### Soluble Films Including Doxepin

This example describes exemplary soluble films for dispensing doxepin. Soluble films, as used herein, are films that substantially dissolve or break down over time when disposed in contact with mucosa, and/or saliva or other bodily fluids. The films may be configured to dissolve or break down over any suitable time period, such as about thirty minutes to about twelve hours, or about two to six hours, among others.

Soluble films may be formed from any suitable composition. In some examples, soluble films may be formed by drying gels. Exemplary gels that may be dried may be formed with any suitable gel compositions listed above in Example 1.

An example of a soluble film contains doxepin at a concentration in the range of 0.1 to 5.0% (w/w). The carrier film comprises pullulan, menthol and aspartame, potassium acesulfame, copper gluconate, polysorbate 80, carrageenan, glyceryl oleate, eucalyptol, methyl alicylate, thymol, locust bean gum, propylene glycol, xanthan gum, and a coloring agent, or a subset of these components.

### EXAMPLE 3

### Foams Including Doxepin

This example describes exemplary foams including doxepin. A foam, as used herein, is a dispersion of gas bubbles in a liquid, solid, or gel. The dispersion may be stable enough to persist in a foam state for any suitable period of time, including about fifteen minutes to twelve hours, among others. Foams may be used for application to local sites.

Foams may include a solvent and various foaming agents, surfactants, emulsifiers, emulsion stabilizers, and/or foam wall thickeners, among others. Exemplary solvents may include water, an alcohol, and/or a mixture of water and an oil. Exemplary foaming agents, surfactants, emulsifiers, and/or emulsion stabilizers may include sodium lauryl sulfate, sucrose monostearate, sucrose distearate, cetyl phosphate, stearic acid, cetyl alcohol, sodium monostearate, cocoamide diethanolamine, lauramide diethanolamine, polypropylene glycol-14-butyl ether, sodium N-methyl N-cocoyl taurate, sodium methyl cocoyl-N-coco-beta-aminobutyric acid, monosodium N-lauryl-1-glutamate, and/or monosodium-N-cocoyl-1-glutamate, among others. Exemplary foam thickeners may include glycerol, sorbitol, hydrogenated starch hydrolysate, and/or the like.

Foams may be formed by any suitable mechanism. In some examples, the foams may be formed as they are dispensed from an aerosol container. Dispensing may be facilitated with an aerosol propellant, such as propane, butane, etc.

### EXAMPLE 4

### Sprays and Aerosols Including Doxepin

This example describes sprays and aerosols including doxepin. Sprays, as used herein are gas-borne solid or liquid particles, drops, and/or streams that can be directed to a surface or an area. Sprays may include particles or drops of any suitable size, generally about 10-20 micrometers or greater in diameter. Aerosols, as used herein, are fine solid or liquid particles suspended in gas. The particles in aerosols may have any suitable diameter, for example, about 1 micrometer to about 20 micrometers. Sprays and/or aerosols may permit application of doxepin to mucosal sites that are difficult to approach through other delivery mechanisms.

Sprays and/or aerosols may be formed by passing a composition including doxepin from a container through a suitable outlet structure. The outlet structure may include an atomizer, an orifice, a channel, and/or the like. The size of the drops or particles formed may be adjusted based on the size and/or shape of the outlet structure, a pressure exerted on the outlet by the composition (for example, from inside the container by a propellant in the container), the rate at which the composition is released form the container, and/or the like.

Sprays and/or aerosols including doxepin may be produced from any suitable composition. The composition may include, for example, a solvent, such as water, an additive to increase the viscosity (such as a polymer, for example, polyethylene glycol), a gelling agent, etc. Alternatively, no solvent may be included. For example, methyl cellulose can be used as a dry powder mixed with doxepin and placed in a spray container, to permit the methyl cellulose/doxepin to be directed as a powder spray from the container to selected sites.

### EXAMPLE 5

### Pastes Including Doxepin

This example describes pastes (and/or ointments or salves) including doxepin. A paste, as used herein, is a soft, plastic (moldable) composition that is semisolid. Pastes/ointments or salves may be used for application to sites of physical irritation and abrasion. A paste may be formed, for example, by mixing a suitable solvent (such as water) with a solid or a very viscous liquid. An exemplary paste for application to a mucosal tissue may be formed by mixing methyl cellulose with water. Alternatively, or in addition, a paste may include colloidal particles, such as colloidal silica, as in gel toothpastes. These colloidal pastes may be aqueous in nature and made from particles that are so small they become suspended in water without being dissolved in water.

### EXAMPLE 6

### Solid Compositions Including Doxepin

This example describes solid compositions including doxepin. Solid compositions may be suitable for application to large areas of mucosal tissue without direct contact to the mucosal tissue by the application method. Solid compositions may be in any suitable form, including a powder, crystals, pellets, capsules, etc. Doxepin may have any suitable concentration or proportion within these solid compositions, including about 0.1 to 50%, among others. The solid compositions may include any suitable vehicle, such as a simple or complex carbohydrate and/or a polymer (for example, polyethylene glycol), among others. Doxepin may be incorporated into the solid compositions by mixing, grinding, encapsulation, co-precipitation, drying a liquid or semi-solid composition, and/or the like.

Solid compositions may be applied by any suitable mechanism. Exemplary mechanisms may include mechanical application (such as with a spoon or spatula), as a powder spray, in association with an insoluble (or soluble) carrier (such as a film or tray), etc.

### EXAMPLE 7

### Insoluble Carriers and/or Barriers

This example describes insoluble carriers and/or barriers that may be used to facilitate application of doxepin to mucosal tissues. Insoluble carriers/barriers, as used herein, are structures (such as films, trays, vessels, etc.) that do not break down and/or become dispersed when exposed to saliva or other bodily fluids for a period of at least four hours. Insoluble carriers may be configured as carriers of doxepin compositions, for example, gels, pastes, foams, solutions, and/or solids including doxepin. Accordingly, these compositions may be placed on and/or in each carrier, to be held in apposition to mucosal tissue. For example, a doxepin composition may be disposed on an insoluble film. Alternatively, or in addition, the insoluble structure may function as a barrier. The barrier may restrict movement of doxepin and/or doxepin compositions away from a site of application and/or may restrict access of bodily fluids such as salive, to the doxepin and/or the doxepin composition.

Any suitable materials may be used to form the carriers/barriers. Exemplary carriers/barriers are films formed of a plastic, such as polyethylene, polypropylene, polyvinyl chloride, a polyester, etc.

The following U.S. patent applications and patents are incorporated by reference: Ser. Nos. 10/728,277; 09/993,383; 4,517,173; 4,572,832; 4,713,243; 4,900,554; 5,137,729; 5,770,559; 5,981,474; 6,159,498; 6,479,074; 6,669,960; and 6,685,917.

Excerpted portions of U.S. Pat. No. 6,685,917, which is incorporated by refernce above, are set out in the Appendix below.

### Appendix—Excerpts From U.S. Pat. No. 6,685,917

Column 1, lines 18-33:

Mucositis is a serious and often very painful disorder involving inflammation of the mucous membrane, with the inflammation often accompanied by infection and/or ulceration. Mucositis can occur at any of the different mucosal sites in the body. A nonlimiting list of examples of locations where mucositis can occur include mucosal sites in the oral cavity, esophagus, gastrointestinal tract, bladder, vagina, rectum, lung, nasal cavity, ear and orbita. Mucositis often develops as a side effect of cancer therapy, and especially as a side effect of chemotherapy and radiation therapy for the treatment of cancer. While cancerous cells are the primary targets of cancer therapies, other cell types can be damaged as well. Exposure to radiation and/or chemotherapeutics often results in significant disruption of cellular integrity in mucosal epithelium, leading to inflammation, infection and/or ulceration at mucosal sites.

Column 2, lines 55-67:

Occurrence of mucositis at mucosal sites other than in the oral cavity in association with chemotherapy or radiation therapy are mechanistically similar to the occurrence of oral mucositis. For example, patients undergoing radiation therapy treatment for non-small cell lung cancer frequently develop esophagitis as a side effect of treatment. Esophagitis in this patient population can impede the progress of cancer treatment.

Given that a large number of patients suffer mucositis annually and patients undergoing cancer therapy often receive multiple cycles of chemotherapy and/or radiation therapy, there is a significant need for improved treatment of mucositis. The present invention is directed to this need.

Column 3, Lines 2-67:

In one aspect, the present invention provides a therapeutic composition for the treatment of mucositis. By treatment of mucositis, it is meant that the therapeutic composition is effective to prevent or reduce the incidence, severity and/or duration of the disease. The therapeutic composition comprises at least one pharmaceutical substance that, as formulated in the therapeutic composition, presents therapeutic effect in mammalian hosts, typically human hosts, for the treatment of mucositis, together with at least one biocompatible polymer that aids delivery of the pharmaceutical substance to the targeted mucosal site. One preferred embodiment of the therapeutic composition includes N-acetylcysteine as the pharmaceutical substance and a polyoxyalkylene block copolymer as the biocompatible polymer.

The therapeutic composition can be made with or without reverse-thermal viscosity behavior. For many applications, reverse-thermal viscosity behavior is beneficial to permit administration in a lower viscosity fluid form that tends to convert to a higher viscosity form following administration as the temperature of the therapeutic composition increases in the body. This also facilitates administration at a refrigerated temperature, which is soothing and refreshing to the host in a number of situations, such as for the treatment of mucosal surfaces in the oral cavity or esophagus. The biocompatible polymer will often be a reverse-thermal gelation polymer capable of imparting the desired reverse-thermal viscosity behavior to the therapeutic composition. Also, the therapeutic composition can be made in a variety of product forms, with different product forms being more desirable for targeting treatment to different mucosal sites. Also, in some applications it is desirable that the reverse-thermal viscosity behavior can include reverse-thermal gelation, in which case the therapeutic composition converts to a gel form as the temperature of the composition is increased from below to above a reverse-thermal gel transition temperature. When the therapeutic composition has reverse-thermal gelation properties, the therapeutic composition will preferably have a reverse-thermal gel transition temperature that is no higher than, and even more preferably lower than, the physiological temperature of the host. Depending upon the specific application, the therapeutic composition could be administered to the host at a cold temperature at which the therapeutic composition is in the form of a flowable medium, or at a temperature at which the therapeutic composition is in the form of a gel. When administered in the form of a gel, the therapeutic composition will often have a thick, pudding-like texture. Inside the body, the gel tends to break down as biological fluids dilute the therapeutic composition. But even with breakdown of the gel, significant amounts of the biocompatible polymer and pharmaceutical substance tend to adhere to mucosal surfaces to promote effective delivery of the pharmaceutical substance to treat the targeted mucosal site.

When treating for oral mucositis, the therapeutic composition is preferably administered in the form of a flowable medium with sufficient fluidity for use as a mouthwash that can be swished in the oral cavity to promote adhesion of the biocompatible polymer, and therefore also the pharmaceutical substance, to mucosal surfaces in the oral cavity. The therapeutic composition will typically include a carrier liquid (also referred to herein as a liquid vehicle), such as water, and the pharmaceutical substance and the biocompatible polymer are each dissolved or suspended in the carrier liquid when the therapeutic composition is in the flowable medium form for introduction into the oral cavity.

Column 5, lines 15-43:

In another aspect, the invention involves a therapeutic composition useful for treatment of mucositis at a mucosal site, with the composition comprising a sulfur-containing antioxidant. Such sulfur-containing anti-oxidants include those in which the sulfur is preferably present in a thiol, thioether, thioester, thiourea, thiocarbamate, disulfide, or sulfonium group. A particularly preferred sulfur-containing antioxidant is N-acetylcysteine.

In another aspect, the present invention involves use of the therapeutic composition, in any form and with any formulation, for treatment of mucositis.

In another aspect, a method is provided for delivering to a mucosal site a pharmaceutical substance for treatment of mucositis at a mucosal site, involving introduction into a host of a therapeutic composition of the invention. In one embodiment, the method involves introducing a therapeutic composition into the host, with the therapeutic composition comprising the pharmaceutical substance and a biocompatible polymer. After the therapeutic composition is introduced into the host, at least a portion of the biocompatible polymer and the pharmaceutical substance adhere to a mucosal surface at the mucosal site.

Column 5. Line 59 Through Column 6, Line 36:

As used herein, “NAC” means N-acetylcysteine.

As used herein, “biocompatible” means not having toxic or injurious effects on biological function in humans.

As used herein, “bioadhesive” means having the ability to adhere to a biological surface such as mucous membranes or other tissues for an extended period of time.

As used herein, “transition temperature” or “gel transition temperature” refers to a temperature at which a material, such as the biocompatible polymer or the therapeutic composition as the case may be, changes physical form from a liquid to a gel, or vice versa.

As used herein, “reverse-thermal gel transition temperature” refers to a temperature at which a material, such as the biocompatible polymer or the therapeutic composition as the case may be, changes physical form from a liquid to a gel as the temperature is increased from below to above the temperature, and changes physical form from a gel to a liquid as the temperature is decreased from above to below the temperature.

As used herein, “thermal gelation property” refers to a property of a material, such as the biocompatible polymer or the therapeutic composition, as the case may be, to change physical form from a liquid to a gel, or vice versa, due to a change in temperature.

As used herein, “reverse-thermal gelation property” refers to a property of a material, such as the biocompatible polymer or the therapeutic composition, as the case may be, to change physical form from a liquid to a gel with increasing temperature.

In one aspect, the present invention provides a therapeutic composition for delivery of mucositis therapeutics to humans, especially for use when bioadhesion and permeability of the oral mucositis therapeutic(s) are desired. The composition comprises at least one, and optionally more than one, mucositis therapeutic and a biocompatible polymer. Each mucositis therapeutic is a pharmaceutical substance that provides a therapeutic effect for at least one of prevention of mucositis and treatment of mucositis, either alone or in combination with other materials. In that regard, the therapeutic effect may be due to the direct action of the pharmaceutical substance of the composition, or may be due to one or more other materials activated by the pharmaceutical substance or for which the pharmaceutical substance is a precursor.

Column 6. Lines 47-61:

The amount of mucositis therapeutic in the therapeutic composition of the present invention varies depending on the nature and potency of the therapeutic. In most situations, however, the amount of oral mucositis therapeutic in the composition will be less than about 20% w/w relative to the total weight of the therapeutic composition.

The therapeutic composition of the present invention provides a delivery system for bioadhesion, permeation, or prolonged and sustained action, of the oral mucositis therapeutic, thereby improving the efficacy of the oral mucositis therapeutic upon topical application to mucosal surfaces, a route that may otherwise be an ineffective means of therapy. Furthermore, the delivery system may reduce the frequency and duration of administration of the mucositis therapeutic as part of a treatment.

Column 7 Lines 9-64:

The therapeutic composition can be in any convenient physical form, but is often preferably in the form of a flowable fluid medium at the time of administration. For example, when treating for oral mucositis, the therapeutic composition is preferably sufficiently fluid in character that it can be accepted in the oral cavity and swished in the manner of a mouthwash. In this situation, the therapeutic composition will typically include as its largest constituent a carrier liquid to impart the flowable fluid properties to the therapeutic composition. In most instances the carrier liquid will be water. The biocompatible polymer and mucositis therapeutic are each dissolved in the carrier liquid or suspended in the carrier liquid as a disperse phase. For example, the therapeutic composition can comprise an aqueous solution of the biocompatible polymer, with the mucositis therapeutic also dissolved in the solution and/or suspended as a precipitate in the solution. Preferably, both of the biocompatible polymer and the mucositis therapeutic are dissolved in the carrier liquid, at least at a temperature at which the therapeutic composition is to be administered to patients. Having the biocompatible polymer and the mucositis therapeutic codissolved in the carrier liquid ensures intimate mixing of the two materials, which promotes adhesion of the mucositis therapeutic to surfaces of the oral cavity along with the biocompatible polymer, thereby effectively using the therapeutic.

Proper selection of the biocompatible polymer is important to enhanced performance of therapeutic composition. In one important embodiment, the biocompatible polymer is selected so that when the biocompatible polymer is incorporated into the therapeutic composition, the rheology of the therapeutic composition is such that the viscosity of the therapeutic composition increases with increasing temperature in the vicinity of physiological temperature, which is typically about 37° C. In this way, the therapeutic composition can be administered as a lower viscosity flowable fluid medium at a cool temperature, and the viscosity of the therapeutic composition will increase as the therapeutic composition is warmed to physiological temperature. In one preferred embodiment for many applications when it is desirable for thetherapeutic composition to exhibit reverse-thermal viscosity behavior, the therapeutic composition exhibits reverse-thermal viscosity behavior over at least some range of temperatures between 1° C. and the physiological temperature of the host (e.g., 37° C. for a human host), and preferably over some range of temperatures between 1° C. and 20° C. The therapeutic composition can then be administered to the host in a lower viscosity form at a reduced temperature, typically lower than 20° C. and more typically form 1° C. to 20° C. Often a refrigerated temperature of from 1° C. to 10° C. and more often a refrigerated temperature of from 2° C. to 8° C. will be used. For example, the therapeutic composition may be introduced into the oral cavity at a temperature of from about 1° C. to about 20° C., and more preferably a temperature of from about 1° C. to about 10° C.

Column 8 Line 20 Through Column 10, Line 4:

Particularly preferred biocompatible polymers, when the composition is to be administered with the biocompatible polymer in solution form dissolved in a solvent, include cellulosic polymers, glycerin, polyethylene glycol and polyoxyalkylene block copolymers.

Reverse-thermal gelation polymers are especially useful for imparting desirable rheological properties to the therapeutic composition. These biocompatible reverse-thermal gelation polymers can be incorporated into the therapeutic composition at concentrations so that the therapeutic composition has reverse-thermal gelation properties, or can be incorporated into the therapeutic composition at a concentration that does not impart reverse-thermal gelation properties to the therapeutic composition, but otherwise provides desired viscosity behavior for a particular application.

As used herein, the terms “reverse-thermal viscosity property” and “reverse-thermal viscosity behavior” each refer to a property of a component or components, and in particular a biocompatible polymer/water combination, to undergo a viscosity increase with increasing temperature across at least some temperature range. A reverse-thermal gelation property is a one type of reverse-thermal viscosity behavior in which a component or components, and in particular a biocompatible polymer/water combination in the therapeutic composition, change from a liquid form to a gel form as the temperature is raised from below to above a reverse-thermal gel transition temperature. “Reverse-thermal gelation polymer” refers to a polymercapable of interacting with a liquid vehicle, and particularly water, so that the polymer/liquid vehicle combination exhibits a reverse-thermal gelation property when the polymer and liquid vehicle are combined in at least some proportion. It should be appreciated that, if desired, a reverse-thermal gelation polymer and water can be incorporated into the therapeutic composition in such proportions that the therapeutic composition does not have a reverse-thermal gelation property, or does not even exhibit any reverse-thermal viscosity behavior. For most situations, however, the presence of reverse-thermal viscosity behavior is preferred.

With reverse-thermal viscosity behavior (which may or may not involve reverse-thermal gelation), the therapeutic composition can be administered to a patient at a cool temperature, as noted above, which provides a beneficial ‘cold’ feeling upon tissue, such as in the oral cavity or esophagus, of the host following administration. Also the therapeutic composition tends to become more viscous, and possibly even gelatinous depending upon the concentration of biocompatible polymer used, as the therapeutic composition warms to physiological temperature, depending upon the rapidity with which the therapeutic composition is diluted by biological fluids. Such reverse-thermal viscosity behavior does tend to promote greater bioadhesion of the biocompatible polymer and the pharmaceutical substance onto mucosal surfaces, leading to longer contact time of the pharmaceutical substance at the targeted mucosal site.

Furthermore, the biocompatible polymer and other components of the therapeutic composition may aid in the permeation of a mucosal therapeutic into the mucosa. For example, permeation into the oral mucosa or across oral mucosal cell membranes may aid in placing the therapeutic agent at additional target sites as well as provide for sustained action of the therapeutic agent within the oral mucosa.

Non-limiting examples of some biocompatible reverse-thermal gelation polymers include certain polyethers (preferably polyoxyalkylene block copolymers with more preferred polyoxyalkylene block copolymers including polyoxyethylene-polyoxypropylene block copolymers referred to herein as POE-POP block copolymers, such as Pluronic™ F68, Pluronic™ F127, Pluronic™ L121, and Pluronic™ L101, and Tetronic™ T1501); certain cellulosic polymers, such as ethylhydroxyethyl cellulose; and certain poly (ether-ester) block copolymers (such as those disclosed in U.S. Pat. No. 5,702,717, the entire contents of which are incorporated by reference herein as if set forth herein in full). Pluronic™ and Tetronic™ are trademarks of BASF Corporation. Furthermore, more than one of these and/or other biocompatible polymers may be included in the therapeutic composition. Also, other polymers and/or other additives may also be included in the therapeutic composition to the extent the inclusion is not inconsistent with the desired characteristics of the therapeutic composition. Furthermore, these polymers may be mixed with other polymers or other additives, such as sugars, to vary the transition temperature, typically in aqueous solutions, at which reverse-thermal gelation occurs.

As will be appreciated, any number of biocompatible polymers may now or hereafter exist that are capable of being used in the therapeutic composition, and such polymers are specifically intended to be within the scope of the present invention when incorporated into the therapeutic composition.

Polyoxyalkylene block copolymers are particularly preferred as biocompatible polymers for use in the therapeutic composition. A polyoxyalkylene block copolymer is a polymer including at least one block (i.e. polymer segment) of a first polyoxyalkylene and at least one block of a second polyoxyalkylene, although other blocks may be present as well. POE-POP block copolymers are one class of preferred polyoxyalkylene block copolymers for use as the biocompatible reverse-thermal gelation polymer in the formulated biocompatible polymer. POE-POP block copolymers include at least one block of a polyoxyethylene and at least one block of a polyoxypropylene, although other blocks may be present as well. The polyoxyethylene block may generally be represented by the formula (C₂H₄O)_{b} when b is an integer. The polyoxypropylene block may generally be represented by the formula (C₃H₆O)ₐ when a is an integer. The polyoxypropylene block could be for example (CH₂CH₂CH₂O)ₐ, or could be CH₃ |(CHCH₂O)a

Several POE-POP block copolymers are known to exhibit reverse-thermal gelation properties, and these polymers are particularly preferred for imparting reverse-thermal viscosity and/or reverse-thermal gelation properties to the therapeutic composition. Examples of POE-POP block copolymers include Pluronic™ F68, Pluronic™ F127, Pluronic™ L121, Pluronic™ L101, and Tetronic™ T1501. Tetronic™ T1501 is one example of a POE-POP block copolymer having at least one polymer segment in addition to the polyoxyethylene and polyoxypropylene segments. Tetronic™ T1501 is reported by BASF Corporation to be a block copolymer including polymer segments, or blocks, of ethylene oxide, propylene oxide and ethylene diamine.

Some preferred POE-POP block copolymers have the formula:
HO(C₂H₄O)_{b}(C₃H₆O)ₐ(C₂H₄O)_{b}H I
which, in the preferred embodiment, has the property of being liquid at ambient or lower temperatures and existing as a semi-solid gel at mammalian body temperatures wherein a and b are integers in the range of 15 to 80 and 50 to 150, respectively. A particularly preferred POE-POP block copolymer for use with the present invention has the following formula:
HO(CH₂CH₂O)_{b}(CH₂(CH₃)CHO)ₐ(CH₂CH₂O)_{b}H II
wherein a and b are integers such that the hydrophobe base represented by (CH₂(CH₃)CHO)ₐ has a molecular weight of about 4,000, as determined by hydroxyl number; the polyoxyethylene chain constituting about 70 percent of the total number of monomeric units in the molecule and where the copolymer has an average molecular weight of about 12,600. Pluronic™ F-127, also known as Poloxamer 407, is such a material. In addition, a structurally similar Pluronic™ F-68 may also be used.
Column 110 Lines 46-64:

When the therapeutic composition exhibits reverse-thermal gelation properties, the amount of biocompatible polymer and the amount of oral mucositis therapeutic agent are typically selected such that the resulting composition has a reverse-thermal gel transition temperature that is not higher than the physiological temperature of the host (e.g., 37° C. for human hosts). In most situations, the reverse-thermal gel transition temperature will be in a range having a lower limit of about 10, more typically about 10° C., sometimes about 20° C. and sometimes even 25° C., and having an upper limit typically of about 40° C., more typically about 37° C. and even more typically about 25° C. Particularly preferred when the therapeutic composition has reverse-thermal gelation properties is for the reverse-thermal gel transition temperature to be in a range of from about 10° C. to about 25° C. In this situation, the reverse-thermal polymer/liquid vehicle combination will be in a liquid form when stored at normal refrigeration storage temperatures of 2° C. to 8° C.

Column 11, Lines 26-50:

The concentration of the biocompatible polymer in the composition will vary depending upon the specific biocompatible polymer and the specific situation. In most situations, however, the biocompatible polymer will comprise from about 1% by weight to about 70% by weight, and more typically from about 5% by weight to about 20% by weight of the therapeutic composition. For example, particularly preferred for use of Pluronic® F-127 in many applications is a range of from about 10% by weight to about 20% by weight of the therapeutic composition.

The therapeutic composition of the present invention can also comprise other additives, including polymer or therapeutic agent stabilizers including sucrose, salts, and pH adjusting agents; preservatives including antioxidants such as butylated hydroxytoluene, antifungals, and antibacterials; and taste masking components. Inclusion of taste masking components is particularly desirable when administration is in the oral cavity, such as for treatment of oral mucositis or esophagitis. Nonlimiting examples of taste masking components include fruit flavorings (and particularly citrus flavorings), mint flavorings, salt, or sugars. In one preferred embodiment, the taste masking component imparts a citrus flavor, and preferably lemon flavor to the composition, such as when the taste masking component comprises lemon juice or a lemon extract.

Column 12, Line 65 Through Column 15, Line 28:

Nonlimiting examples of mucositis therapeutics that may be used to make the therapeutic composition of the present invention include antioxidants, antibacterials, antiinflammatories, anesthetics, analgesics, proteins, peptides and cytokines, including those currently available or later developed. Preferably the mucositis therapeutic is selected from the group consisting of antioxidants. More preferably the antioxidant is selected from the group consisting of sulfur-containing antioxidants or vitamin antioxidants, with sulfur-containing antioxidants generally being more preferred. Even more preferably, the sulfur-containing antioxidant includes sulfur in at least one constituent group selected from thiol, thioether, thioester, thiourea, thiocarbamate, disulfide and sulfonium, with thiol-containing antioxidants (also referred to as sulfhydryl-containing antioxidants) being particularly preferred. Some examples of preferred thiol-containing antioxidants include N-acetylcysteine (NAC) and glutathione. Other examples of preferred sulfur-containing antioxidants include S-carboxymethylcysteine and methylmethionine sulfonium chloride.

In an especially preferred embodiment, the sulfur-containing antioxidants are precursors for biosynthesis of glutathione in the host, such as by providing cysteine or a precursor for cysteine for glutathione synthesis. In this way, the mucosal therapeutic promotes the production of glutathione. Examples of antioxidants that are precursors for glutathione biosynthesis include NAC, procysteine, lipoic acid, s-allyl cysteine, and methylmethionine sulfonium chloride. In one preferred embodiment the mucositis therapeutic is NAC.

Examples of vitamin antioxidants include vitamin E, vitamin E mimetics, vitamin E analogs, vitamin C, and vitamin A. Particularly preferred in the vitamin class of antioxidants are water soluble vitamin forms of vitamin E, including Trolox and vitamin E TGPS (d-.alpha.-tocopherol polyethylene glycol 1000 succinate).

The action and selection of the antioxidant are not limited by the above description as many antioxidants may have a multitude of actions and thus fall under several classes of antioxidants or several classes of therapeutic agents. For example, NAC may directly scavenge free radicals extracellularly and provide cysteine intracellularly as a precursor for intracellular scavenging of free radicals via glutathione biosynthesis and regulation of glutathione-dependent antioxidative enzymes. Another example includes curcumin, which, in addition to its antioxidative action, possesses anti-inflammatory and antiproliferative actions that are beneficial in preventing or alleviating the clinical course of oral mucositis. In addition to therapeutic action, the antioxidant selected may exert other beneficial effects as a component of the therapeutic composition including bioadhesion as in the case of lipid soluble forms of vitamin E and penetration enhancement as in the case of lipoic acid, curcumin, and vitamin E TGPS.

The amount of mucosal therapeutic included in the therapeutic composition of the present invention varies depending on the nature and potency of the particular therapeutic. Typically, however, the amount of mucosal therapeutic present in the therapeutic composition is in a range having a lower limit typically of about 0.001%, more typically about 0.01%, and even more typically about 0.1% by weight of the therapeutic composition, and having an upper limit of typically about 50%, more typically about 25%, and even more typically about 10% by weight of the therapeutic composition.

The therapeutic composition of the present invention may be administered to a host (patient) to achieve any desired effect in the clinical outcome of the targeted mucositis. Preferably the host is a mammal, and more preferably a human. The therapeutic composition can be administered in a variety of forms adapted to the chosen route of administration.

When treating for oral mucositis, the therapeutic composition is contacted with the oral mucosa in the oral cavity. Administration in this situation can include, for example, use of a mouthwash, spray, lollipop or other product form of the formulation. Preferably, the mode of administering the therapeutic composition for treating oral mucositis is a mouthwash which, after being swished in the mouth, may then be spit out or, more preferably, swallowed in order to coat both mucosal surfaces in the mouth and in the esophagus, as well as provide systemic effects upon gastrointestinal absorption.

The therapeutic composition is typically prepared in water or a saline solution. Under ordinary conditions of storage and use, these preparations can also contain a preservative to prevent the growth of microorganisms. For oral mucositis applications, the therapeutic composition typically is a fluid, i.e., in a liquid form, to the extent that it is palatable and thus, easily tolerated, by the often nauseous cancer patient. The therapeutic composition can be stable under the conditions of manufacture and storage and preferably preserved against the contaminating action of microorganisms such as bacteria and fungi. The carrier liquid can be a solvent of dispersion medium containing, for example, water, ethanol, polyol (e.g., glycerol, propylene glycol, and liquid polyethylene glycol, and the like), and suitable mixtures thereof. The proper fluidity can be maintained, for example, by maintaining the temperature of the therapeutic composition having reverse-thermal gelation properties below the transition temperature. The prevention of the action of microorganisms can be brought about by various antibacterial and antifungal agents, for example, parabens, chlorobutanol, phenol, sorbic acid, thimerosal, benzoic acid, alcohol, benzalkonium chloride and the like. In many cases, it will be preferable to include isotonic agents, e.g., sugars, phosphate buffers, sodium benzoate, sodium chloride, or mixtures thereof.

In many situations, it will be desirable for the therapeutic composition to be in the form of a flowable medium when introduced into the host for treatment of a mucosal site. This will often be the case for example for oral mucositis applications in which the therapeutic composition is to be administered as a refrigerated mouthwash. In one preferred embodiment, the therapeutic composition has a relatively low viscosity when the therapeutic composition is at a temperature for introduction into the host for treatment. In this embodiment, the viscosity of the therapeutic composition when introduced into the host is no larger than 60 cP (centipoises), and more preferably no larger than 50 cP. Because the therapeutic composition is typically administered at a reduced temperature, in this embodiment, the therapeutic composition will preferably have a viscosity at 2° C. of no larger than 60 cP and more preferably no larger than 50 cP. When the therapeutic composition exhibits reverse-thermal viscosity behavior, the viscosity of the therapeutic composition will preferably exhibit an increase in viscosity from a viscosity of no larger than 60 cP (and more preferably no larger than 50 cP) to a viscosity of at least 70 cP, or even 80 cp or more (and more preferably even larger) as the temperature of the therapeutic composition is increased over at least some range of temperatures between 1° C. and the physiological temperature of the host (e.g., 37° C. for a human host). When the therapeutic composition has reverse-thermal gelation properties, the viscosity will often increase to a level of 90 cp, or even 100 cP or more with an increase in temperature from below to above the reverse-thermal gel transition temperature.

In some situations when treating for oral mucositis, it will be desirable to specifically target sublingual mucosal surfaces. In this situation, the therapeutic composition can be sublingually placed, such as in the form of a tablet, patch or film. In one preferred sublingual application, the therapeutic composition is already in the form of a gel when sublingually placed, and the gel then dissipates as it is diluted with biological fluids. In this situation, the administered gel can have a thick, pudding-like texture and can be spooned or squeezed from a tube into the sublingual location. In this situation, when administered, the therapeutic composition will typically have a viscosity of at least 70 cP, and more typically a viscosity of at least 80 cP, at least 90 cP or even at least 100 cP.

For oral mucositis applications when the therapeutic composition has reverse-thermal gelation properties, the therapeutic composition can be used as a mouthwash at a temperature below the reverse-thermal gel transition temperature, whereupon the therapeutic composition will ordinarily become more viscous or even gelatinous as it warms inside the mouth. Not all aspects of the invention when treating for oral mucositis are so limited, however. For example, in some instances the therapeutic composition may not become more viscous or gelatinous inside the mouth of the host, but the biocompatible polymer will still provide some protection to the oral mucositis therapeutic and enable contact and permeation of the mucositis therapeutic within the oral mucosa.

1-54. (canceled)

55. A gelling composition useful for administering doxepin to mucosal tissue for relief of pain associated with mucositis, the composition comprising:
doxepin, at a pharmaceutically effective concentration for relief of pain associated with mucositis; and
a gelling agent, comprising poloxamer; and
water, as a major component of the composition.

56. The composition of claim 55, comprising from 0.1% to 5% by weight of the doxepin.

57. The composition of claim 56, comprising a drug effective for treatment of the mucositis.

58. The composition of claim 57, wherein the drug is N-acetylcysteine.

59. The composition of claim 58, wherein, the poloxamer is a polyoxyalkylene block copolymer of a type and at a concentration that interacts with the water to impart reverse-thermal viscosity behavior to the composition, whereby the viscosity of the composition exhibits the reverse-thermal viscosity behavior over at least some range of temperatures between 1° C. and 37° C.

60. The composition of claim 59, comprising from 5% to 20% by weight of the polyoxyalkylene block copolymer.

61. The composition of claim 60, wherein the polyoxyalkylene block copolymer is poloxamer 407.

62. The composition of claim 61, comprising from 0.1% to 1% by weight of the doxepin.

63. A method for treating a mucosal region for pain relief associated with mucositis, the method comprising topical treatment of the mucosal region with the composition of claim 55.

64. A method for treating a mucosal region for pain relief associated with mucositis, the method comprising topical treatment of the mucosal region with the composition of claim 57.

65. A composition useful for combined treatment for mucositis and for relief of pain associated with the mucositis, the composition comprising:
N-acetylcysteine, in an amount effective for treatment for the mucositis; and
doxepin, at a pharmaceutically effective concentration for treatment for relief of the pain associated with the mucositis; and
water, as a major component.

66. The composition of claim 65, comprising from 0.1 to 5% by weight of the doxepin.

67. The composition of claim 66, comprising from 0.1 to 20% by weight of the N-acetylcysteine.

68. The composition of claim 67, comprising from 5 to 20% by weight of a polyoxyalkylene block copolymer.

69. The composition of claim 68, wherein the polyoxyalkylene block copolymer is of a type and at a concentration that interacts with the water to impart reverse-thermal viscosity behavior to the composition, whereby the viscosity of the composition exhibits the reverse-thermal viscosity behavior over at least some range of temperatures between 1° C. and 37° C.

70. The composition of claim 69, wherein the polyoxyalkylene block copolymer is poloxamer 407.

71. The composition of claim 70, comprising from 0.1 to 1% by weight of the doxepin.

72. A method for combined treatment for mucositis at a mucosal region in a patient's mouth and for relief of pain associated with the mucositis, the method comprising
introducing the composition of claim 65 into the mouth of the patient to contact the mucosal region.

73. The method of claim 72, wherein the mucositis is a side effect of the patient undergoing cancer therapy treatment.

74. The method of claim 73, wherein the cancer therapy treatment comprises one or both of radiation therapy and chemotherapy.

75. A method for combined treating for mucositis at a mucosal region in a patient's mouth and for relief of pain associated with the mucositis, the method comprising introducing the composition of claim 71 into the mouth of the patient to contact the mucosal region.
